(19) United States
(12) Patent Application Publication
(10) Pub. No.: US 2007/0137642 A1
(43) Pub. Date: Jun. 21, 2007

(54) Infant stomach band to protect from injury

(76) Inventor: Lydia Anita Tillett, Antioch, TN (US)

Correspondence Address: LYDIA ANITA KINNARD, 3936 ROCK CREEK DRIVE, ANTIOCH, TN, 37013, US

(21) Appl. No.: 11/440,611

(22) Filed: May 24, 2006

Related U.S. Application Data
(60) Provisional application No. 60/750,544, filed on Dec. 15, 2005.

(51) Int. Cl. A61F 5/24

(52) U.S. Cl. 128/96.1

(57) ABSTRACT

Disclosed herein is an infant stomach band to protect the infant from injury. This band has particular usefulness in helping to prevent hernias in infants and preventing protruding belly buttons in infants. The band can have a especially useful applications for babies having colic.

This Patent Application filed for the invention by Lydia A. Kinnard, of Antioch, Tenn. 37013-5623 of a “Infant Stomach Band To Protect From Injury.”

A portion of the disclosure of this patent document contains material that is subject to copyright protection. The copyright owner has no obligation to the facsimile reproduction by anyone of the patent document or the patent disclosure, as it appears in the U.S. Patent and Trademark Office patent file or records, but otherwise reserves all copyright rights whatsoever.

All patents and publications described or discussed herein are hereby incorporated by reference in their entireties.

### BACKGROUND OF THE INVENTION

This present invention relates generally to a band used to protect infants from injury. More specifically, the present invention relates to a band designed to prevent hernias in infants and help prevent protruding belly buttons in infants. The current invention can have especially useful application for infants having colic.

There have been numerous advancements in medical science, including medical science directed at the improvement of the health and well being of infants. These advancements have been focused on both proactive and reactive treatments. However, infant healthcare can be especially difficult since infants do not have the capacity to specifically communicate to the caregivers the health issues that they are experiencing. For example, obviously an infant cannot specifically articulate to a doctor nurse, or caregiver the specific areas experiencing pain or any specific description of the type of sensation of the pain. This difficulty leads to a lot of crying by the infant and reactive care by the caregiver. This reactive care is normally in the form of food, additional clothing, diaper changes, and the like. This care to infant is normally guesswork on the caregiver's part. As such, the infant can experience stress and manifest that stress in long emotional outbursts of crying. These outbursts can lead to stress-related symptoms within the infant, such as a hernia.

Much of the prior art attempts to address hernias in infants have revolved around externally applied medicine for hernias. For example, U.S. Pat. No. 6,921,547 is directed at such a device. This device specifically calls for a dosage unit in powder form contained within a fabric pouch applied to the navel of a baby suffering from a hernia. As such, this patent is reactive to the already medically-diagnosed issue, mainly a hernia and the infant still experiences the pain and requires time to heal in order to recover from the physical ailment.

What is needed then is a preventive device and/or medical treatment in which to prevent hernias in infants and can prevent the protruding belly buttons in infants. This needed device can preferably be disposable or non-disposable and varying adjustments used to correspond to the various sizes of infants.

### BRIEF SUMMARY OF THE INVENTION

Disclosed herein is an infant stomach band to protect the infant from injury. This band has particular usefulness in helping to prevent hernias in infants and preventing protruding belly buttons in infants. The band can have a especially useful applications for babies having colic.

The band preferably includes an expanded middle section that substantially corresponds to the lower abdomen of an infant. A first and second armature extends from the expanded middle section wherein opposite ends of the armature include attachment devices used to attach the free end of the armatures together around the infant.

The infant band can also include a secondary attachment on the expanded section that interfaces with the diaper of the infant to the band in place. The attachment devices at the distal ends of the bands can be various attachment devices known, such as Velcro, adhesives, and the like.

It is therefore a general object of the present invention to provide an infant stomach band.

Another object of the present invention is to provide an infant stomach band to protect an infant from injury, such as a strain-related injury.

Another object of the present invention is to provide an infant stomach band to protect an infant from a hernia.

Another object of the present invention is to provide an infant stomach band to prevent the infant from developing a protruding belly button.

Another object of the present invention is to provide an adjustable band used to secure the lower abdomen of an infant.

Other and further objects, features and advantages of the present invention will be readily apparent to those skilled in the art upon reading of the following disclosure when taken in conjunction with the accompanying drawings.

### BRIEF DESCRIPTION OF SEVERAL VIEWS OF THE DRAWINGS

FIG. 1 is a perspective view of an infant band made in accordance with the current disclosure.

FIG. 2 is a cross-section taken along line A-A of FIG. 1.

FIG. 3 is a perspective view of an alternative embodiment of an infant band made in accordance with the current disclosure.

FIG. 4 is a black perspective view of the infant band shown in FIG. 3

### DETAILED DESCRIPTION OF THE INVENTION

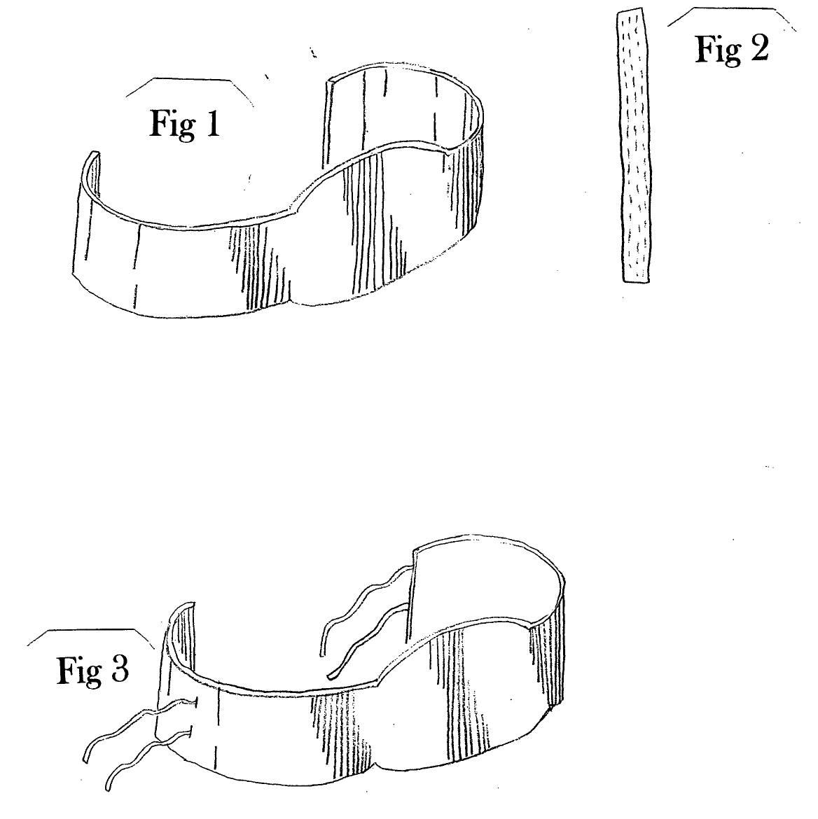

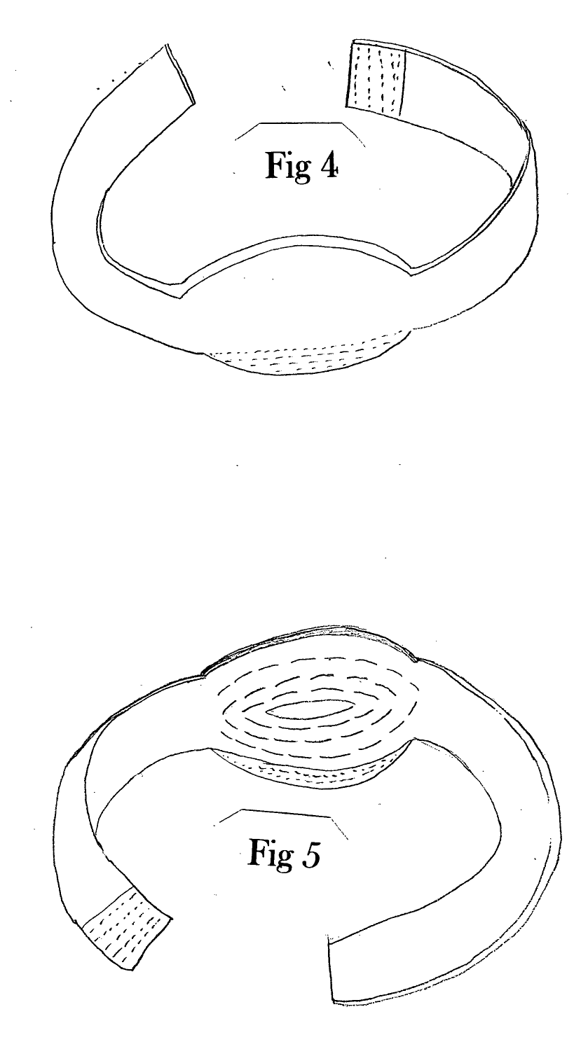

Referring generally to FIG. 1-4, an infant band is shown and generally designated by the numeral 10. The band 10 includes an expanded section 12 and first and second armatures 14 and 16 extending from the expanded section 12. The expanded section 12 includes a width that is preferably wider than the armatures 14 and 16. The expanded section can include an attachment device 20 positioned thereon to facilitate the attachment of the band to the clothing and/or diaper of an infant. This attachment device 20 can be materials known in the art to secure two items together, such as Velcro, adhesives, and the like. Preferably the attachment 20 is designed such that it can be repeatedly used to secure the band 10 to the clothing and/or diapers of the infant.

The first and second armatures 14 and 16 can include a securing device 22 used to remove ably attach the distal ends 15 and 17 of the armatures 14 and 16, respectively. The securing device 22 can be a single piece, or multiple pieces, known in the art to secure two items together. Examples are Velcro adhesives. And the like. Preferably the securing device 22 can be multiple times to secure the distal ends 15 and 17 of the armatures 14 and 16 respectively. This secure device 22 facilitates a proper fit of the band 10 around the waist of an infant.

Preferably the band 10 compromises a cloth type material 24 covering an elastic material 26. The elastic material 26 can provide the resistance needed to help prevent the hernia and/or bulging belly buttons in infants. The cloth material 24 helps facilitate the attachment between the band 10 and the diaper and/or clothing of the infant while providing protection to the infant from the elastic material 26. The cloth material 24 is preferably stretchable and breathable while being a soft and comfortable material. Additional padding can be included beneath the cloth material 24 and can be preferably positioned between a cloth material and elastic material 26 to provide comfort to the infant.

Ideally the band can be used once the umbilical cord has fallen off of an infant and that area is fully healed. This can take approximately 2-3 weeks for proper healing to allow use of the band.

In a preferred embodiment the band 10 compromises disposable materials substantially similar to the diaper materials used for infants' diapers. The band 10 can include absorbent qualities as present in those in diapers while maintaining the predetermined geometrical shape to help prevent the hernia injuries and bulging belly button areas in infants.

In an alternate preferred embodiment the band 10 can be composed of washable materials such that any soiling of the band 10 can be cleaned in normal washing. This alternate embodiment will be composed of stronger attachment devices and securing 20 and 22 respectively to withstand the cleaning and washing of the band 10. The elastic material 26 could be removable from within the cloth material 24 to facilitate this washing and cleaning.

Still yet another alternative embodiment of the band 10 compromises a cloth material that is composed of substantially all cotton. This cotton is designed in such a way to provide the required pressure on the abdomen of the infant to help prevent the hernia injury. As such, in this embodiment the substantially all cotton band would be washable without the need of removal of any additional materials from within or special washing techniques.

The band 10 can have various types of designs to enhance the aesthetic appearance of the band. For example, these designs can be gender based as well as specific activity based. These designs would normally be positioned external to the band 10 and can be positioned on the cloth material 24 to facilitate the aesthetic look of the band 10.

Thus, although there have been described particular embodiments of the present invention of a new and useful Infant Stomach Band To Protect From Injury, it is not intended that such references be construed as limitations upon the scope of this invention except as set fort in the following claims.

What is claimed is:

1. All inventive material disclosed herein.

2. All novel features disclosed herein.

3. A band for the prevention of hernias in children.

4. A band designed to fit around the abdominal region of an infant, the band comprising an expanded section shaped to substantially mirror the abdominal region of the infant and first and second armatures extending from the expanded section, wherein the armatures include a securing device on the ends distal from the expanded section.

5. The band of claim 4, wherein the expanded section includes an attachment device to secure the band to the clothing of an infant.

6. A band of claim 4, wherein the expanded section includes an attachment device designed to attach the band to a diaper worn by the infant.

7. A band for the abdominal region of an infant, the band compromises an elastic material designed to apply pressure to the abdominal region of the infant to restrict bulges in the abdominal region.

8. A band of claim 7, wherein the band applies pressure to reduce the presence of hernias in infants.
